(19) United States
(12) Patent Application Publication
(10) Pub. No.: US 2015/0051535 A1
(43) Pub. Date: Feb. 19, 2015

(54) HIGH-PRESSURE PERFLUOROCARBON WOUND BANDAGE

(71) Applicant: Mark G. FONTENOT, Lafayette, LA (US)

(72) Inventor: Mark G. FONTENOT, Lafayette, LA (US)

(21) Appl. No.: 14/204,576

(22) Filed: Mar. 11, 2014

Related U.S. Application Data
(60) Provisional application No. 61/777,866, filed on Mar. 12, 2013.

(51) Int. Cl. A61M 35/00

(52) U.S. Cl. CPC A61M 35/00
USPC 604/23

(57) ABSTRACT

Systems and method comprise forming a cavity adjacent a wound. Formation of the cavity comprises applying a bandage to an area adjacent the wound. A topical hyperbaric oxygen emulsion (THOE) is applied to the wound, and the THOE comprises a perfluorocarbon (PFC). The cavity holds a pressure when the THOE is applied, wherein the pressure increases oxygen saturation of the wound and reduces oxygen diffusion from the THOE.

### RELATED APPLICATION

This application claims the benefit of U.S. patent application Ser. No. 61/777,866, filed Mar. 12, 2013.

### TECHNICAL FIELD

The embodiments described herein relate generally to a method and apparatus for delivering oxygen to tissue.

### BACKGROUND

The literature is replete with references describing use of perfluorocarbons for blood transfusion, preservation of tissues, and delivery of perfluorocarbon-oxygen formulations to tissue. In view of the conventional methods and systems in the literature, there is a need for systems and methods for creating a closed non-compliant polymeric chamber enclosing a wound to topically deliver oxygen to tissues via perfluorocarbon-based solutions in a high-pressure wound enclosure.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 shows a bandage, under an embodiment.

FIG. 2 shows initiation of the process of applying THOE to a wound, under the embodiment.

FIG. 3 shows the wound with THOE applied, under the embodiment.

FIG. 4 shows the wound with the THOE and bandage applied, under the embodiment.

FIG. 5 is a top view of the wound with the THOE and bandage applied, under the embodiment.

FIG. 6 shows the bandage-wound (BW) cavity with the oxygen outgassing from the THOE, under the embodiment.

FIG. 7 shows a bandage with a relief valve, under an alternative embodiment.

FIG. 8 shows the wound with the bandage applied, under the alternative embodiment.

FIG. 9 shows the THOE canister reversibly coupled to the inflow valve of the bandage, under the alternative embodiment.

FIG. 10 shows dispensing of the THOE into the BW cavity created by the bandage, under the alternative embodiment.

FIG. 11 shows a syringe reversibly coupled to the inflow valve and the contents of the syringe being dispensed into the BW cavity to rinse the wound, under an embodiment.

FIG. 12 shows the application of pressure via massage (arrow) to remove the emulsion from the BW cavity, under an embodiment.

### DETAILED DESCRIPTION

Systems and method comprise forming a cavity adjacent a wound. Formation of the cavity comprises applying a bandage to an area adjacent the wound. A topical hyperbaric oxygen emulsion (THOE) is applied to the wound, and the THOE comprises a perfluorocarbon (PFC). The cavity holds a pressure when the THOE is applied, wherein the pressure increases oxygen saturation of the wound and reduces oxygen diffusion from the THOE.

Embodiments described herein relate to systems and methods for creating a closed non-compliant polymeric chamber enclosing a wound to topically deliver oxygen to tissues via perfluorocarbon based solutions in a high pressure wound enclosure. More particularly, embodiments described herein include a topical hyperbaric oxygen emulsion (THOE) comprising a dispersed phase of perfluorocarbon (PFC) droplets encapsulated within an aqueous continuous phase. The PFC used in an embodiment is perflurordecalin (PFD) and is selected for its high oxygen solubility, chemical inertness, and biocompatibility, but the embodiment is not limited to PFD. The THOE is packaged in a pressurized bladder canister containing 50 grams of THOE, for example. The pressurized canister ensures that oxygen remains in solubilized form in the THOE, and does not outgas within the container. During manufacturing, the THOE is pumped into the bladder of the canister, resulting in an intra-canister pressure of approximately 230 psig. Since the canister is pressurized, the oxygen remains in the THOE. When the THOE is dispensed from the canister onto a wound or into a wound-bandage cavity, oxygen reaches the tissues through diffusion or other transport mechanisms.

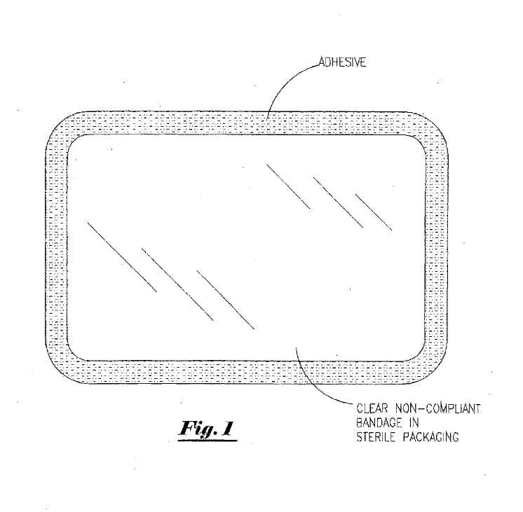

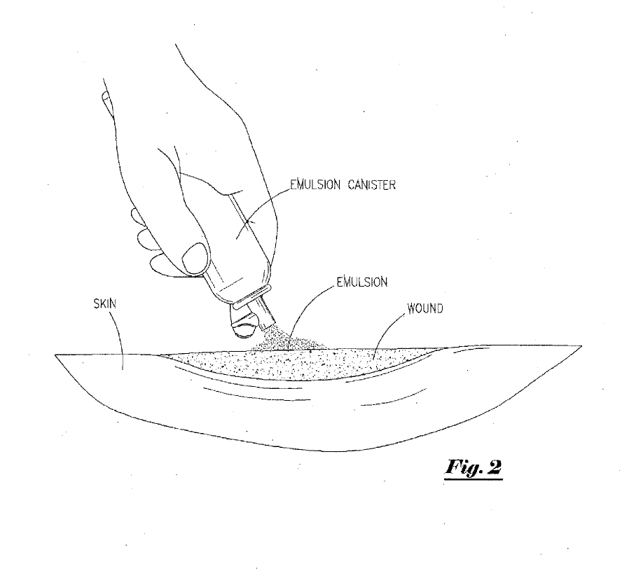

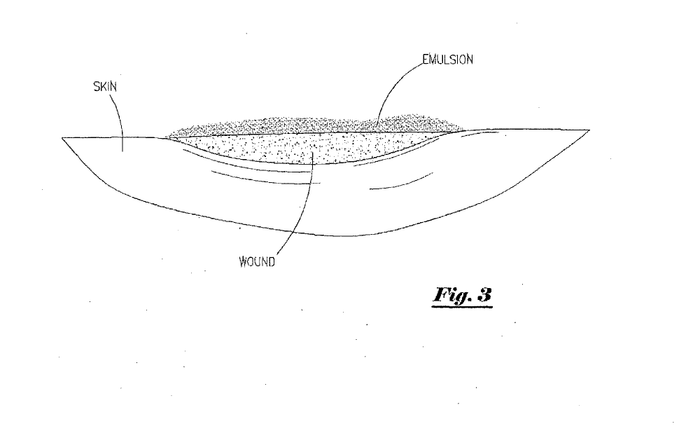

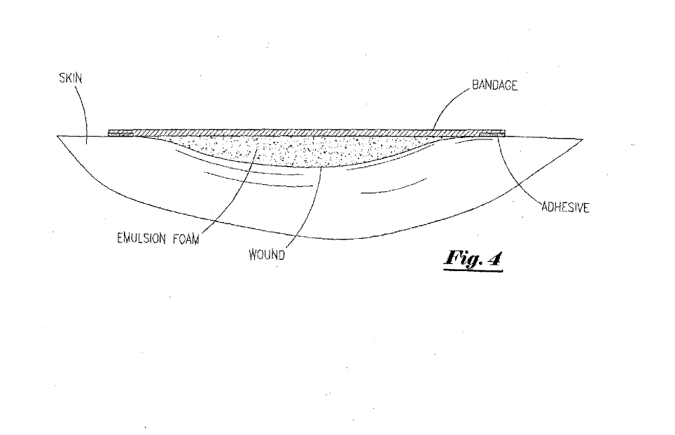

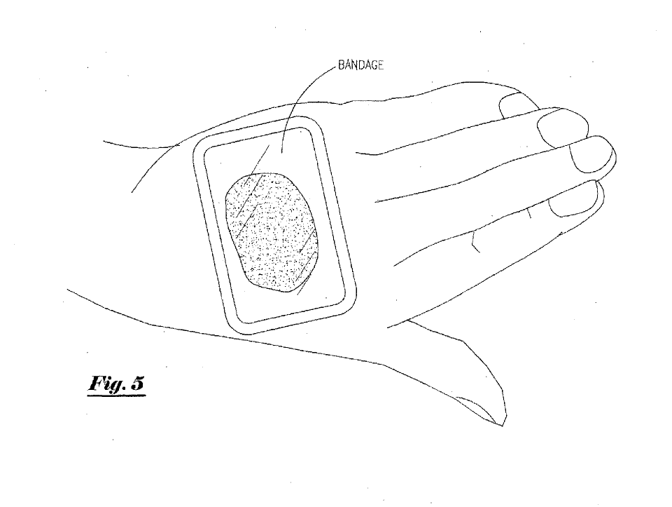

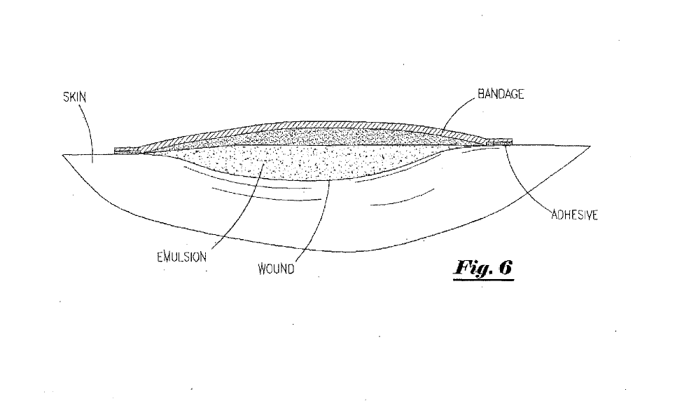

In the case of THOE, embodiments may include multiple devices and combinations thereof to enable an improved and prolonged transport of topical oxygen into tissues. Under one embodiment, a clear non-compliant bandage with a rim of adhesive circumferentially placed on one side of the bandage is placed over a wound that has been treated with THOE. FIG. 1 shows a bandage, under an embodiment. FIG. 2 shows initiation of the process of applying THOE to a wound, under an embodiment. FIG. 3 shows the wound with THOE applied, under an embodiment. FIG. 4 shows the wound with the THOE and bandage applied, under an embodiment. FIG. 5 is a top view of the wound with the THOE and bandage applied, under an embodiment. As the oxygen outgases from the THOE, the internal pressure of the bandage-wound (BW) cavity increases, thus reducing the rate of oxygen diffusion from the THOE and increasing the oxygen saturation of the wound secondary to the increased internal pressure in the BW cavity. FIG. 6 shows the BW cavity with the oxygen outgassing from the THOE, under an embodiment. Thus, the bandage of an embodiment decreases the frequency of dispensing the THOE over the wound, improves delivery of oxygen to the wound with prolonged outgassing of the THOE, and reduces the cost of treating the wound with THOE.

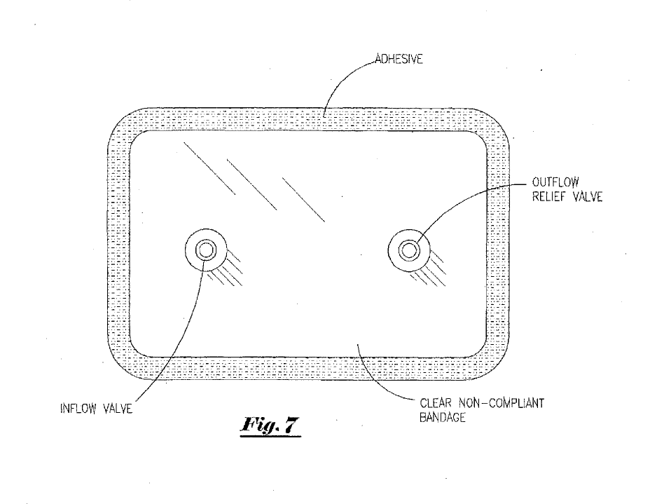

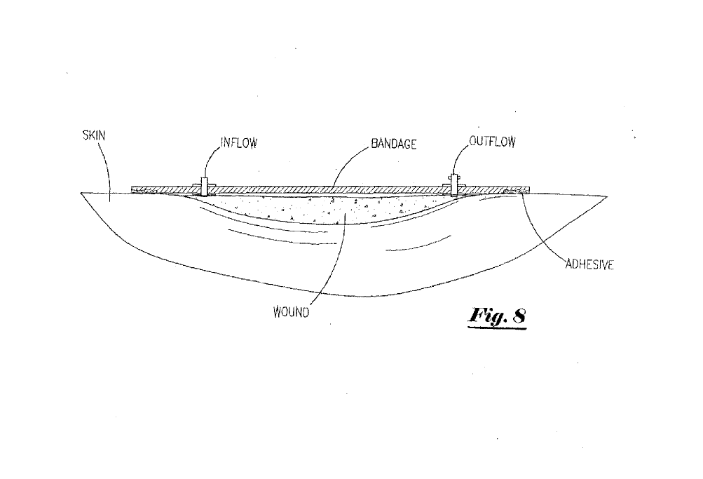

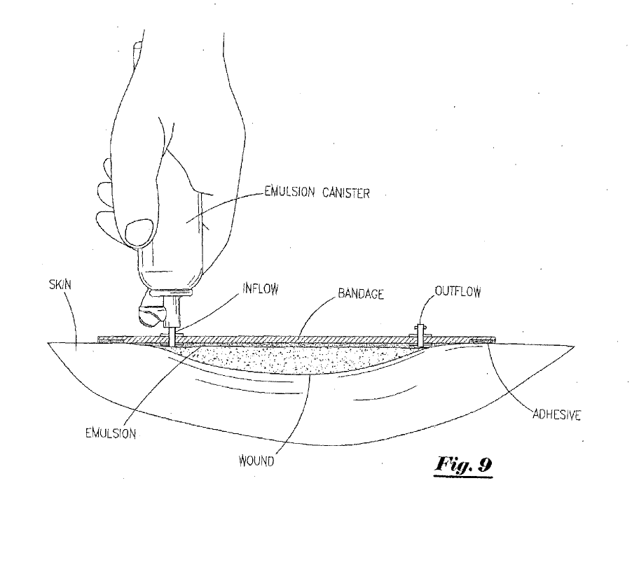

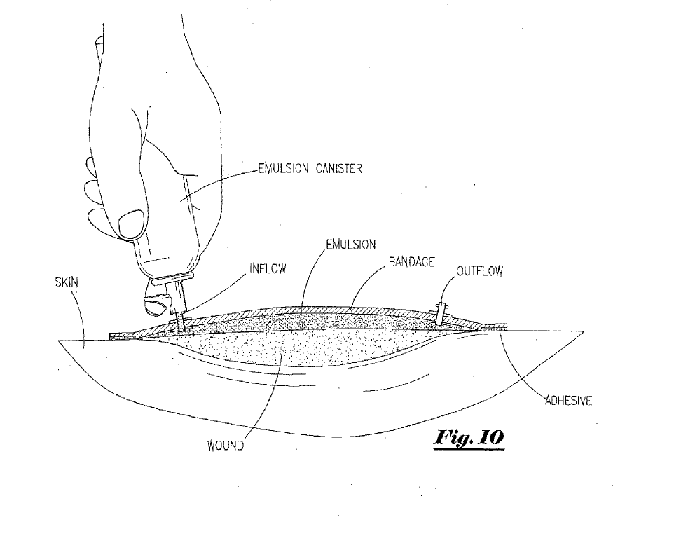

In an alternative embodiment, a clear non-compliant bandage with a rim of adhesive circumferentially placed on one side of the bandage is placed over the wound. FIG. 7 shows a bandage with a relief valve, under an alternative embodiment. FIG. 8 shows the wound with the bandage applied, under the alternative embodiment. The bandage of this alternative embodiment has an inflow valve and outflow valve. FIG. 9 shows the THOE canister reversibly coupled to the inflow valve of the bandage, under the alternative embodiment. FIG. 10 shows dispensing of the THOE into the BW cavity created by the bandage, under the alternative embodiment. As the oxygen outgases from the THOE, the internal pressure of the BW cavity increases, thus reducing the rate of oxygen diffusion from the THOE and increasing and prolonging oxygen saturation of the wound secondary to the increased internal pressure of the BW cavity.

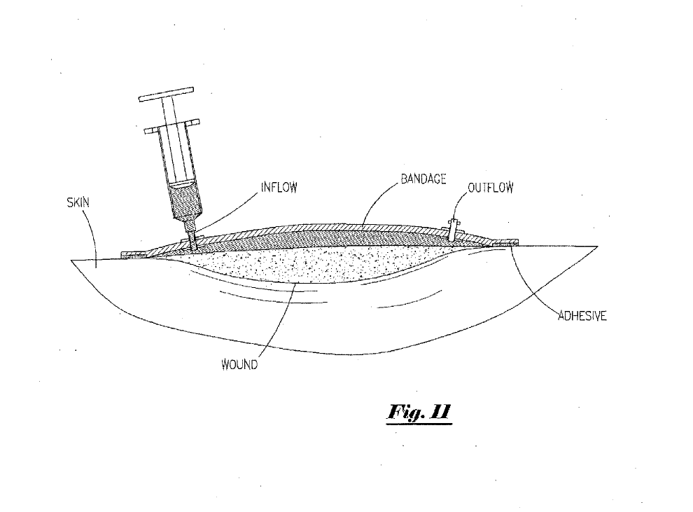

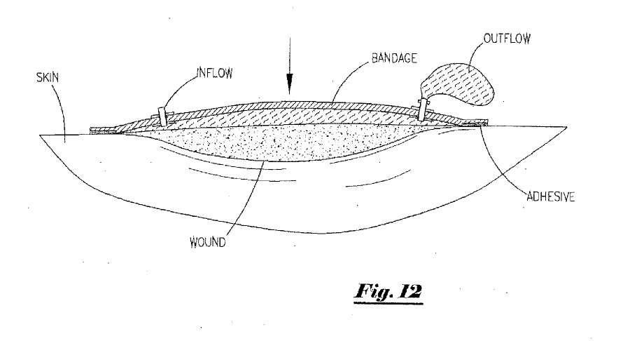

If, at some point, the outgassed emulsion is to be removed from the wound, then the outflow valve may be opened and the emulsion massaged from the BW cavity. FIG. 12 shows the application of pressure via massage (arrow) to remove the emulsion from the BW cavity, under an embodiment. Furthermore, the cavity can be rinsed by reversibly coupling a syringe to the inflow valve and dispensing the contents of the syringe into the BW cavity. FIG. 11 shows a syringe reversibly coupled to the inflow valve and the contents of the syringe being dispensed into the BW cavity to rinse the wound, under an embodiment. A number of formulations can be dispensed into the BW cavity (e.g., Dermacyn, antibiotics, normal saline, PFD, etc.) via a syringe coupled to the inflow valve.

In another alternative embodiment, the canister that is reversibly coupled to the bandage includes pure PFD saturated with, for example, oxygen (e.g., 10 atmospheres oxygen). The PFD-oxygen formulation is dispensed into the BW cavity and over the wound, thus covering the wound with oxygen saturated PFD. The oxygen will outgas from the PFD leaving the wound covered and saturated with PFD. The BW cavity pressure would increase secondary to outgassing of the oxygen from the PFD-oxygen solution; however, the oxygen partial pressure would be greater in the BW cavity, and this oxygen pressure gradient between the BW cavity oxygen partial pressure and the PFD saturated wound would allow oxygen to move easier into the tissue at a higher level when compared to tissue alone and with a PFD component. In other words, the PFD-saturated tissue has a greater oxygen carrying capacity when compared to tissue alone or hypoxic tissue in the case of a wound.

In addition to the methods described herein, the PFD of an embodiment can be used in conjunction with other techniques and equipment. For example, EpiFlo is a conventional device that consists of a small, silent, disposable, oxygen concentrator and a 60″ long sterile cannula (tube). It may be used with any fully occlusive sterile wound dressing to continuously blanket the wound with near 100% oxygen. The patient is free to ambulate and can continue with normal daily living activities while being treated 24 hours per day. EpiFlo may under an embodiment be worn near the wound beneath clothing without impairing its operation.

In an alternative embodiment, PFD is dispensed into the BW cavity via a syringe (see FIG. 11), or the canister dispensing either THOE or PFD-oxygen solution. The objective is to saturate the wound with PFD, then couple the EpiFlo to the inflow valve of the bandage. The transfer of the 100% oxygen to blanket the wound is more efficient since the wound and tissues are saturated with PFD (PFD has a solubility coefficient that is approximately 500 to 1,000 higher than biologic tissues or water) allowing oxygen to move easier into tissue.

Yet another alternative embodiment comprises a non-compliant polymeric bandage that can have a printed circuit board (PCB) etched on the surface wherein leads are coupled to the skin. A waveform (e.g., DC, AC, DC with an AC offset, etc.) may be introduced to drive the PFD or THOE into the skin and further saturate the wound and skin with PFD and oxygen. Under yet another embodiment, surface mount near infrared (IR) light emitting diodes (LED) can be mounted to the bandage and used to expose the wound to near IR. Such exposure has been shown to accelerate wound healing.

In the description above, numerous specific details are introduced to provide a thorough understanding of, and enabling description for, embodiments of the systems and methods. One skilled in the relevant art, however, will recognize that these embodiments can be practiced without one or more of the specific details, or with other components, systems, etc. In other instances, well-known structures or operations are not shown, or are not described in detail, to avoid obscuring aspects of the disclosed embodiments.

Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods is not intended to be exhaustive or to limit the systems and methods described to the precise form disclosed. While specific embodiments of, and examples for, the systems and methods are described herein for illustrative purposes, various equivalent modifications are possible within the scope of other systems and methods, as those skilled in the relevant art will recognize. The teachings of the systems and methods provided herein can be applied to other processing systems and methods, not only for the systems and methods described above.

The elements and acts of the various embodiments described above can be combined to provide further embodiments. These and other changes can be made to the systems and methods in light of the above detailed description.

What is claimed is:

1. A system comprising:
a topical hyperbaric oxygen emulsion (THOE) comprising a dispersed phase of perfluorocarbon (PFC); and
a bandage comprising a region of adhesive around a perimeter of the bandage;
wherein the bandage, when placed adjacent a wound, forms a cavity that includes the wound and holds a pressure within the cavity when the THOE is applied to the wound;
wherein the pressure increases oxygen saturation of the wound and reduces oxygen diffusion rate from the THOE.

2. A method comprising:
forming a cavity adjacent a wound, wherein the forming of the cavity comprises applying a bandage to an area adjacent the wound; and
applying a topical hyperbaric oxygen emulsion (THOE) to the wound, wherein the THOE comprises a perfluorocarbon (PFC);
wherein the cavity holds a pressure when the THOE is applied, wherein the pressure increases oxygen saturation of the wound and reduces oxygen diffusion from the THOE.
